(19) United States
(12) Patent Application Publication
(10) Pub. No.: US 2006/0205962 A1
(43) Pub. Date: Sep. 14, 2006

(54) Olefin epoxidation process, a catalyst for use in the process, a carrier for use in making the catalyst, and a process for making the carrier

(76) Inventors: Leonid Isaakovich Rubinstein, Houston, TX (US); Randall Clayton Yeates, Sugar Land, TX (US)

Correspondence Address: SHELL OIL COMPANY, P O BOX 2463, HOUSTON, TX, 772522463, US

(21) Appl. No.: 11/357,881

(22) Filed: Feb. 17, 2006

Related U.S. Application Data
(60) Provisional application No. 60/654,487, filed on Feb. 21, 2005.

(51) Int. Cl. C07D 301/10; B01J 23/50; B01J 23/48

(52) U.S. Cl. 549/534; 502/348

(57) ABSTRACT

A carrier that may be used in the manufacture of an olefin epoxidation catalyst is provided that is obtained from a process involving the acid digestion of aluminum metal. Also provided is an olefin epoxidation catalyst comprising a silver component deposited on the carrier. Also provided is a process for the epoxidation of an olefin employing the catalyst and a process for producing a 1,2-diol, a 1,2-diol ether, or an alkanolamine employing the olefin oxide.

This application claims the benefit of U.S. Provisional Application No. 60/654,487 filed Feb. 21, 2005 the entire disclosure of which is hereby incorporated by reference.

### FIELD OF THE INVENTION

The invention relates to a catalyst, a carrier for use in making the catalyst, and methods for making the catalyst and the carrier. The invention also relates to a process for the epoxidation of an olefin employing the catalyst. The invention also relates to methods of using the olefin oxide so produced for making a 1,2-diol, a 1,2-diol ether, or an alkanolamine.

### BACKGROUND OF THE INVENTION

In olefin epoxidation, feed containing an olefin and an oxygen source is contacted with a catalyst under epoxidation conditions. The olefin is reacted with oxygen to form an olefin oxide. A product mix results that contains olefin oxide and typically unreacted feed and combustion products, including carbon dioxide. The olefin oxide, thus produced, may be reacted with water to form a 1,2-diol, with an alcohol to form a 1,2-diol ether, or with an amine to form an alkanolamine. Thus, 1,2-diols, 1,2-diol ethers, and alkanolamines may be produced in a multi-step process initially comprising olefin epoxidation and then the conversion of the formed olefin oxide with water, an alcohol, or an amine.

Olefin epoxidation catalysts are generally comprised of silver, usually with one or more additional elements deposited therewith, on a carrier, typically containing alpha-alumina. Such catalysts are commonly prepared by a method involving impregnating or coating the carrier particles with a solution comprising a silver component. The carrier is commonly prepared by forming particles from a dough or paste comprising the carrier material or a precursor thereof and calcining the particles at a high temperature, commonly at a temperature in excess of 900° C.

The performance of the silver containing catalyst may be assessed on the basis of selectivity, activity, and stability of operation in the olefin epoxidation. The selectivity is the molar fraction of the converted olefin yielding the desired olefin oxide. As the catalyst ages, the fraction of olefin reacted normally decreases with time. To maintain a desired constant level of olefin oxide production, the temperature of the reaction generally is increased. However, increasing the temperature causes the selectivity of the reaction to the desired olefin oxide to decrease. In addition, the equipment used in the reactor typically may tolerate temperatures only up to a certain level. Thus, it may become necessary to terminate the reaction when the reaction temperature reaches a temperature inappropriate for the reactor. Thus, the longer the selectivity may be maintained at a high level and the epoxidation may be performed at an acceptably low reaction temperature while maintaining an acceptable level of olefin oxide production, the longer the catalyst charge may be kept in the reactor and the more product is obtained.

Over the years, much effort has been devoted to improving the performance of olefin epoxidation catalysts. Such efforts have been directed toward improvements to initial activity and selectivity, and to improved stability performance, that is the resistance of the catalyst against aging-related performance decline. In certain instances, improvements have been sought by altering the compositions of the catalysts. In other instances, improvements have been sought by altering the processes for preparing the catalysts, including altering the composition of the carrier and the process for obtaining the carrier.

Reflecting these efforts, modern silver-based catalysts may comprise, in addition to silver, one or more high-selectivity dopants, such as components comprising rhenium, tungsten, chromium, or molybdenum. High-selectivity catalysts are disclosed, for example, in U.S. Pat. No. 4,761,394 and U.S. Pat. No. 4,766,105, herein incorporated by reference. U.S. Pat. No. 4,766,105 and U.S. Pat. No. 4,761,394 disclose that rhenium may be employed as a further component in the silver containing catalyst with the effect that the initial selectivity of the olefin epoxidation is increased. EP-A-352850 also teaches that the then newly developed catalysts, comprising silver supported on alumina carrier, promoted with alkali metal and rhenium components have a very high selectivity.

With regard to efforts to improve the process of preparing the catalysts, U.S. Pat. No. 6,368,998, which is incorporated herein by reference, shows that washing the carrier with water, prior to the deposition of silver, leads to catalysts that have improved initial performance properties.

Not withstanding the improvements already achieved, there is a desire to further improve the performance of olefin epoxidation catalysts.

### SUMMARY OF THE INVENTION

The present invention provides a carrier comprising alpha-alumina, which carrier is obtainable from a process comprising acid digestion of aluminum. The present invention also provides a method of preparing a carrier comprising acid digesting aluminum to obtain transition alumina; forming a paste comprising the transition alumina; and forming carrier particles comprising transition alumina from the paste. In preferred embodiments, the method comprises an additional step of calcining the shaped carrier particles at a temperature between 900° C. and 1400° C.

In preferred embodiments, the process comprises acid digesting aluminum to obtain an alumina sol; forming transition alumina powder from the alumina sol; forming a paste with the transition alumina powder; forming carrier particles comprising transition alumina from the paste; and calcining the carrier particles at a temperature between 900° C. and 1400° C. In preferred embodiments, the paste is formed from a mixture comprising alumina sol and the transition alumina powder. The present invention also provides a carrier comprising alpha-alumina, which carrier is obtainable by a process in accordance with this invention.

The present invention also provides a catalyst for the epoxidation of an olefin comprising a silver component deposited on a carrier comprising alpha-alumina, wherein the carrier is obtainable from a process in accordance with this invention. In preferred embodiments, the carrier is a fluoride mineralized carrier. In preferred embodiments, the carrier comprises a particulate matrix having a morphology characterizable as lamellar. In preferred embodiments, the catalyst additionally comprises a high selectivity dopant. The present invention also provides a process for the epoxidation of an olefin comprising the steps of contacting a feed comprising an olefin and oxygen with a catalyst comprising a silver component deposited on a carrier comprising alpha-alumina; and producing a product mix comprising an olefin oxide, wherein the carrier is obtainable from a process in accordance with this invention. In preferred embodiments, the olefin comprises ethylene. The present invention also provides a process for the production of a 1,2-diol, a 1,2-diol ether or an alkanolamine comprising converting an olefin oxide into the 1,2-diol, the 1,2-diol ether or the alkanolamine wherein the olefin oxide has been obtained by a process for the epoxidation of an olefin in accordance with the present invention.

### DETAILED DESCRIPTION OF THE INVENTION

The catalyst carriers of the present invention are prepared by a process that involves the acid digestion of aluminum. Catalysts prepared in accordance with this invention, using a carrier obtained from a process in which aluminum is subjected to acid digestion, exhibit an unexpected improvement in performance in olefin epoxidation relative to catalysts, which, while otherwise identical, were prepared using a different carrier. In preferred embodiments, the carrier of the present invention is a fluorine mineralized carrier.

The improved performance achieved as a result of the present invention is apparent from one or more of improved initial activity, improved initial selectivity, improved activity stability, and improved selectivity stability. Initial selectivity is meant to be the maximum selectivity that is achieved in the initial phase of the use of the catalyst wherein the catalyst slowly but steadily exhibits an increasing selectivity until the selectivity approaches a maximum selectivity, which is termed the initial selectivity. The initial selectivity is usually, but not necessarily, reached before cumulative olefin oxide production over the catalyst bed has amounted to, for example, 0.15 kTon/m³ of catalyst bed, in particular to 0.1 kTon/m³ of catalyst bed.

The catalysts of the present invention comprise carriers prepared in accordance with the present invention having deposited thereon a silver component. In preferred embodiments, amongst others, the catalyst additionally comprises a high-selectivity dopant. In preferred embodiments, amongst others, the catalyst additionally comprises a Group IA metal component. In preferred embodiments, amongst others, the catalyst additionally comprises a rhenium component or a rhenium component and rhenium co-promoter.

The process for the epoxidation of an olefin of the present invention comprises the steps of contacting a feed comprising an olefin and oxygen with such a catalyst and producing a product mix comprising an olefin oxide.

As disclosed hereinbefore, the forming of the carrier particles involves the acid digestion of aluminum metal. The aluminum is preferably in the form of aluminum wire, platelets, or other shape or form that affords a greater potential for the uniform digestion of the aluminum.

The preferred digestion media comprise an aqueous acid of sufficient strength to avoid a state of zero charge in the digestion system. Accordingly, the preferred digestion media may have a pH lower than about 5, in particular in the range of from 1 to 4, when measured at 20° C. Preferred acids also have anions that decompose or vaporize during subsequent drying or calcining steps. Accordingly, organic acids are preferred. Acceptable acids include acetic, citric, nitric, and phosphoric acids. Acetic acid is particularly preferred.

The concentration of the acid in the digestion system is not of critical importance. However, at high acid concentrations, the reaction rate may be excessive, possibly resulting in large quantities of hydrogen that may overpressure the digestion vessel. At low concentrations, the reaction rate may be too slow for economical reasons. Thus, acid concentrations ranging from about 0.5 to 10 wt. %, in particular from about 2 to 4 wt. %, are typical. Acetic acid at a concentration of 3 wt. % is particularly preferred.

The time required for digestion may vary based on the dimension of the aluminum source and acid strength and concentration. Typically, the digestion is carried out for a period ranging from 15 to 40 hours. The digestion is desirably carried out at a temperature sufficiently high to provide adequate viscosity to achieve digestion and sufficiently low to avoid hazards. Thus, digestion is conveniently carried out at temperatures ranging from about 50° C. to about 110° C., in particular from about 75° C. to about 90° C.

Once all the metal has been digested, in various embodiments, it may be desirable to increase the crystallinity of the alumina sol obtainable from the acid digestion. The crystallinity may be increased by stirring the sol while maintaining the temperature in the range of 50-110° C., in particular 75-90° C. for a period of 1 to 5 days, in particular 2 to 3 days.

The alumina sol will commonly contain about 10 wt. % alumina (dry basis), 3 wt. % acetic acid, and deionized water as the remainder; however, alumina sols with different concentrations and compositions are contemplated. The alumina sol is dried to obtain a transition alumina powder. The drying process is not particularly critical and a variety of procedures are acceptably employed. Spray drying as well as drying in bulk followed by grinding are acceptable methods. Spray drying at a temperature in the range of 300-400° C. is suitable.

The transition alumina powder is thereafter formed into carrier particles. The forming of the carrier particles may comprise shaping and those shapes known in the art, including spheres and cylinders, are contemplated by the present invention. In preferred embodiments, the transition alumina powder is extruded to form the carrier particles. In such preferred embodiments, the transition alumina powder is conveniently converted into a dough or paste prior to being extruded. The transition alumina is commonly mixed with compositions that aid the formation of the paste and/or aid the extrusion. A preferred such composition is alumina sol, desirably the alumina sol prepared as described above as an intermediate to the transition alumina powder. Desirably, the weight ratio of transition alumina powder to alumina sol is as much as 1000:500, in particular as much as 1000:600, more in particular as much as 1000:650, and even more in particular as much as 1000:700. Desirably, the weight ratio of transition alumina powder to alumina sol is as low as 1000:850, in particular as low as 1000:800, and more in particular as low as 1000:750. A particularly desired weight ratio of transition alumina powder to alumina sol is 1000:730. It is believed that the extrusion benefit of the alumina sol is due, at least in part, to its acting as a peptizing agent. Other acceptable extrusion aids include, but are not limited to, acids, including nitric, acetic, and citric; organic extrusion aids, including methocel, PVA, and steric alcohols; and combinations thereof. Binding agents may also be used during the formation of the carrier particles.

The carrier particles of the present invention are subjected to a high temperature calcination, generally in excess of about 900° C., typically in excess of 1000° C., in particular in excess of about 1100° C., and often as much as 1400° C., in particular as much as 1300° C., and more in particular as much as 1200° C. to convert transition alumina into alpha-alumina. While the calcination must be carried out at a temperature sufficient to cause formation of alpha-alumina, the present invention is otherwise independent of the manner by which the calcination is conducted. Thus, variations in calcining known in the art, such as holding at one temperature for a certain period of time and then raising the temperature to a second temperature over the course of a second period of time, are contemplated by the present invention. Calcination is conducted for a time sufficient to achieve a desired surface area, with longer times resulting in particles with a lower surface area. Two hours is a typical time period for the calcination process.

Prior to such high temperature calcination, it is contemplated that the carrier particles may be subjected to a low temperature drying step and/or a low temperature calcination. Such might be the case, for example, when the carrier is manufactured in one location or by one entity but the final catalyst is manufactured in another location or by another entity. Such a low temperature drying step and/or low temperature calcination may be by any methods known in the art, and the temperature and length of time of such processes may vary. For example, low temperature drying between 110° C. and 140° C. for over ten hours is desirable as is drying at 190° C. for six to seven hours. Acceptable low temperature calcination may also be conducted at a temperature between 400° C. and 750° C., desirably between 550° C. and 700° C. for a period of between 30 minutes and 5 hours, desirably between 1 hours and 2 hours.

In certain embodiments, the process for preparing the carriers of the present invention also comprises incorporating in the carrier a fluorine-containing species, as further described hereinafter, which is capable of liberating fluoride when the combination is calcined, and calcining the combination. Such carriers are conveniently referred to as fluoride-mineralized carriers. Preferably, any calcination conducted after the incorporation of fluorine is conducted at less than about 1200° C., more preferably less than about 1100° C. Preferably, any such calcination is conducted at greater than about 900° C., more preferably greater than about 1000° C. If the temperature is sufficiently greater than 1200° C., an excessive amount of fluoride may escape the carrier.

Within these limitations, the manner by which the fluorine-containing species is introduced is not limited, and those methods known in the art for incorporating a fluorine-containing species into a carrier (and those fluoride-mineralized carriers obtained therefrom) may be used for the present invention. For example, U.S. Pat. No. 3,950,507 and U.S. Pat. No. 4,379,134 disclose methods for making fluoride-mineralized carriers and are hereby incorporated by reference.

The present invention is also not limited with respect to the point in the process for manufacturing the carrier when the fluorine-containing species is incorporated. Thus, the fluorine-containing species may be physically combined with transition alumina powder prior to the formation of the carrier particles. For example, the transition alumina powder may be treated with a solution containing a fluorine-containing species. The combination may be co-mulled and then formed into carrier particles. The fluorine may also be incorporated into the carrier particles prior to high temperature calcination, for example, by vacuum impregnation. Any combination of solvent and fluorine-containing species that results in the presence of fluoride ions in solution may be used in accordance with such a method.

In another suitable method, a fluorine-containing species may be added to carrier particles after the formation of alpha-alumina. In such a method, the fluorine-containing species may conveniently be incorporated in the same manner as silver and other promoters, e.g., by impregnation, typically vacuum impregnation. The carrier particles may thereafter be subjected to calcination, preferably at less than about 1200° C.

In certain embodiments, the carriers may have, and preferably do have, a particulate matrix having a morphology characterizable as lamellar or platelet-type, which terms are used interchangeably. As such, particles having in at least one direction a size greater than about 0.1 micrometers have at least one substantially flat major surface. Such particles may have two or more flat major surfaces. In typical embodiments of this invention, carriers may be used which have said platelet-type structure and which have been prepared by fluoride-mineralization, for example as described herein.

Fluorine-containing species that may be used in accordance with this invention are those species that when incorporated into a carrier in accordance with this invention are capable of liberating fluoride, typically in the form of hydrogen fluoride, when calcined, preferably at less than about 1200° C. Preferred fluorine-containing species are capable of liberating fluoride when calcining is conducted at a temperature of from about 900° C. to about 1200° C. Such fluorine-containing species known in the art may be used in accordance with this invention. Suitable fluorine-containing species include organic and inorganic species. Suitable fluorine-containing species include ionic, covalent, and polar covalent compounds. Suitable fluorine-containing species include F₂, aluminum trifluoride, ammonium fluoride, hydrofluoric acid, and dichlorodifluoromethane.

The fluorine-containing species is typically used in an amount such that a catalyst comprising silver deposited on the fluoride-mineralized carrier, when used in a process for the epoxidation of an olefin as defined in connection with this invention, exhibits a selectivity that is greater than a comparable catalyst deposited on an otherwise identical, non-fluoride-mineralized carrier that does not have a lamellar or platelet-type morphology, when used in an otherwise identical process. Typically, the amount of fluorine-containing species added to the carrier is at least about 0.1 percent by weight and typically no greater than about 5.0 percent by weight, calculated as the weight of elemental fluorine used relative to the weight of the carrier material to which the fluorine-containing species is being incorporated. Preferably, the fluorine-containing species is used in an amount no less than about 0.2 percent by weight, more preferably no less than about 0.25 percent by weight. Preferably, the fluorine-containing species is used in an amount no more than about 3.0 percent by weight, more preferably no more than about 2.5 percent by weight. These amounts refer to the amount of the species as initially added and do not necessarily reflect the amount of any species that may ultimately be present in the finished carrier.

Other than being as described above, the carriers that may be used in accordance with this invention are not generally limited. Typically, suitable carriers comprise at least 85 percent by weight, more typically at least 90 percent by weight, in particular at least 95 percent by weight alpha-alumina, frequently up to 99.9 percent by weight alpha-alumina, based on the weight of the carrier. The carrier may additionally comprise, silica, alkali metal, for example sodium and/or potassium, and/or alkaline earth metal, for example calcium and/or magnesium.

Suitable carriers are also not limited with respect to surface area, water absorption, or other properties. The surface area of the carrier may suitably be at least 0.1 m²/g, preferably at least 0.3 m²/g, more preferably at least 0.5 m²/g, and in particular at least 0.6 m²/g, relative to the weight of the carrier; and the surface area may suitably be at most 10 m²/g, preferably at most 5 m²/g, and in particular at most 3 m²/g, relative to the weight of the carrier. “Surface area” as used herein is understood to relate to the surface area as determined by the B.E.T. (Brunauer, Emmett and Teller) method as described in Journal of the American Chemical Society 60 (1938) pp. 309-316. High surface area carriers, in particular when they are alpha-alumina carriers optionally comprising in addition silica, alkali metal and/or alkaline earth metal, provide improved performance and stability of operation. However, when the surface area is very large, carriers may have lower crush strength.

The water absorption of the carrier may suitably be at least 0.2 g/g, preferably at least 0.3 g/g, relative to the weight of the carrier. The water absorption of the carrier may suitably be at most 0.8 g/g, preferably at most 0.7 g/g, relative to the weight of the carrier. Higher water absorption may be in favor in view of a more efficient deposition of silver and further elements, if any, on the carrier by impregnation. However, at higher water absorptions, the carrier, or the catalyst made therefrom, may have lower crush strength. As used herein, water absorption is deemed to have been measured in accordance with ASTM C20, and water absorption is expressed as the weight of the water that may be absorbed into the pores of the carrier, relative to the weight of the carrier.

In accordance with the present invention, the catalyst comprises a silver component deposited on a carrier prepared in accordance with the present invention. The catalyst may additionally comprise, and preferably does comprise, a high-selectivity dopant. The catalyst may additionally comprise, and preferably does comprise, a Group IA metal component.

The catalyst comprises silver as a catalytically active component. Appreciable catalytic activity is typically obtained by employing silver in an amount of at least 10 g/kg, calculated as the weight of the element relative to the weight of the catalyst. Preferably, the catalyst comprises silver in a quantity of from 50 to 500 g/kg, more preferably from 100 to 400 g/kg, for example 105 g/kg, or 120 g/kg, or 190 g/kg, or 250 g/kg, or 350 g/kg.

The catalyst may comprise, in addition to silver, one or more high-selectivity dopants. Catalysts comprising a high-selectivity dopant are known from U.S. Pat. No. 4,761,394 and U.S. Pat. No. 4,766,105, which are incorporated herein by reference. The high-selectivity dopants may comprise, for example, components comprising one or more of rhenium, molybdenum, chromium, and tungsten. The high-selectivity dopants may be present in a total quantity of from 0.01 to 500 mmole/kg, calculated as the element (for example, rhenium, molybdenum, tungsten, and/or chromium) on the total catalyst. Rhenium, molybdenum, chromium, or tungsten may suitably be provided as an oxide or as an oxyanion, for example, as a perrhenate, molybdate, and tungstate, in salt or acid form. The high-selectivity dopants may be employed in the invention in a quantity sufficient to provide a catalyst having a content of high-selectivity dopant as disclosed herein. Of special preference are catalysts that comprise a rhenium component, and more preferably also a rhenium co-promoter, in addition to silver. Rhenium co-promoters are selected from tungsten, molybdenum, chromium, sulfur, phosphorus, boron, compounds thereof, and mixtures thereof.

When the catalyst comprises a rhenium component, rhenium is typically present in a quantity of at least 0.1 mmole/kg, more typically at least 0.5 mmole/kg, and preferably at least 1 mmole/kg, in particular at least 1.5 mmole/kg, calculated as the quantity of the element relative to the weight of the catalyst. Rhenium is typically present in a quantity of at most 5 mmole/kg, preferably at most 3 mmole/kg, more preferably at most 2 mmole/kg, and in particular at most 1.5 mmole/kg. Again, the form in which rhenium is provided to the carrier is not material to the invention. For example, rhenium may suitably be provided as an oxide or as an oxyanion, for example, as a rhenate or perrhenate, in salt or acid form.

If present, preferred amounts of the rhenium co-promoter are from 0.1 to 30 mmole/kg, based on the total amount of the relevant elements, i.e., tungsten, molybdenum, chromium, sulfur, phosphorus and/or boron, relative to the weight of the catalyst. The form in which the rhenium co-promoter is provided to the carrier is not material to the invention. For example, the rhenium co-promoter may suitably be provided as an oxide or as an oxyanion, in salt or acid form.

Suitably, the catalyst may also comprise a Group IA metal component. The Group IA metal component typically comprises one or more of lithium, potassium, rubidium, and cesium. Preferably the Group IA metal component is lithium, potassium and/or cesium. Most preferably, the Group IA metal component comprises cesium or cesium in combination with lithium. Typically, the Group IA metal component is present in the catalyst in a quantity of from 0.01 to 100 mmole/kg, more typically from 0.50 to 50 mmole/kg, more typically from 1 to 20 mmole/kg, calculated as the total quantity of the element relative to the weight of the catalyst. The form in which the Group IA metal is provided to the carrier is not material to the invention. For example, the Group IA metal may suitably be provided as a hydroxide or salt.

As used herein, the quantity of Group IA metal present in the catalyst is deemed to be the quantity in so far as it may be extracted from the catalyst with de-ionized water at 100° C. The extraction method involves extracting a 10-gram sample of the catalyst three times by heating it in 20 mL portions of de-ionized water for 5 minutes at 100° C. and determining in the combined extracts the relevant metals by using a known method, for example atomic absorption spectroscopy.

The preparation of the catalysts, including methods for incorporating silver, high-selectivity dopant, and Group IA metal is known in the art and the known methods are applicable to the preparation of the catalyst that may be used in accordance with the present invention. Methods of preparing the catalyst include impregnating the carrier with a silver compound and performing a reduction to form metallic silver particles. Reference may be made, for example, to U.S. Pat. No. 5,380,697, U.S. Pat. No. 5,739,075, EP-A-266015, U.S. Pat. No. 6,368,998, WO-00/15333, WO-00/15334 and WO-00/15335, which are incorporated herein by reference.

The reduction of cationic silver to metallic silver may be accomplished during a step in which the catalyst is dried, so that the reduction as such does not require a separate process step. This may be the case if the impregnation solution comprises a reducing agent, for example, an oxalate. Such a drying step is suitably carried out at a temperature of at most 300° C., preferably at most 280° C., more preferably at most 260° C., and suitably at a temperature of at least 200° C., preferably at least 210° C., more preferably at least 220° C., suitably for a period of time of at least 1 minute, preferably at least 2 minutes, and suitably for a period of time of at most 60 minutes, preferably at most 20 minutes, more preferably at most 15 minutes, and more preferably at most 10 minutes.

Although the present epoxidation process may be carried out in many ways, it is preferred to carry it out as a gas phase process, i.e., a process in which the feed is contacted in the gas phase with the catalyst which is present as a solid material, typically in a fixed bed under epoxidation conditions. Epoxidation conditions are those combinations of conditions, notably temperature and pressure, under which epoxidation will occur. Generally, the process is carried out as a continuous process, such as the typical commercial processes involving fixed-bed, tubular reactors.

The typical commercial reactor has a plurality of elongated tubes typically situated parallel to each other. While the size and number of tubes may vary from reactor to reactor, a typical tube used in a commercial reactor will have a length between 4 and 15 meters and an internal diameter between 1 and 7 centimeters. Suitably, the internal diameter is sufficient to accommodate the catalyst. Frequently, in commercial scale operations, the process of the invention may involve a quantity of catalyst which is at least 10 kg, for example at least 20 kg, frequently in the range of from 10² to 10⁷ kg, more frequently in the range of from 10³ to 10⁶ kg.

The olefin used in the present epoxidation process may be any olefin, such as an aromatic olefin, for example styrene, or a di-olefin, whether conjugated or not, for example 1,9-decadiene or 1,3-butadiene. A mixture of olefins may also be used. Typically, the olefin is a mono-olefin, for example 2-butene or isobutene. Preferably, the olefin is a mono-α-olefin, for example 1-butene or propylene. The most preferred olefin is ethylene.

The olefin concentration in the feed may be selected within a wide range. Typically, the olefin concentration in the feed will be at most 80 mole-%, relative to the total feed. Desirably, it will be in the range of from 0.5 to 70 mole-%, in particular from 1 to 60 mole-%, on the same basis. As used herein, the feed is considered to be the composition that is contacted with the catalyst.

The present epoxidation process may be air-based or oxygen-based, see “Kirk-Othmer Encyclopedia of Chemical Technology”, 3^{rd} edition, Volume 9, 1980, pp. 445-447. In the air-based process, air or air enriched with oxygen is employed as the source of the oxidizing agent while in the oxygen-based processes high-purity (typically at least 95 mole-%) oxygen is employed as the source of the oxidizing agent. Presently, most epoxidation plants are oxygen-based and this is a preferred embodiment of the present invention.

The oxygen concentration in the feed may be selected within a wide range. However, in practice, oxygen is generally applied at a concentration that avoids the flammable regime. Typically, the concentration of oxygen applied will be within the range of from 1 to 15 mole-%, more typically from 2 to 12 mole-% of the total feed.

In order to remain outside the flammable regime, the concentration of oxygen in the feed may be lowered as the concentration of the olefin is increased. The actual safe operating ranges depend, along with the feed composition, on the reaction conditions, such as the reaction temperature and the pressure.

A reaction modifier may be present in the feed for increasing the selectivity, suppressing the undesirable oxidation of olefin or olefin oxide to carbon dioxide and water, relative to the desired formation of olefin oxide. Many organic compounds, especially organic halides and organic nitrogen compounds, may be employed as the reaction modifier. Nitrogen oxides, hydrazine, hydroxylamine or ammonia may be employed as well. It is frequently considered that under the operating conditions of olefin epoxidation the nitrogen containing reaction modifiers are precursors of nitrates or nitrites, i.e. they are so-called nitrate- or nitrite-forming compounds (cf. e.g. EP-A-3642 and U.S. Pat. No. 4,822,900, which are incorporated herein by reference).

Organic halides are the preferred reaction modifiers, in particular organic bromides, and more in particular organic chlorides. Preferred organic halides are chlorohydrocarbons or bromohydrocarbons and are preferably selected from the group of methyl chloride, ethyl chloride, ethylene dichloride, ethylene dibromide, vinyl chloride, or a mixture thereof. The most preferred organic halides are ethyl chloride and ethylene dichloride.

Suitable nitrogen oxides are of the general formula NOₓ wherein x is in the range of from 1 to 2, and include for example NO, N₂O₃ and N₂O₄. Suitable organic nitrogen compounds are nitro compounds, nitroso compounds, amines, nitrates and nitrites, for example nitromethane, 1-nitropropane or 2-nitropropane. In preferred embodiments, nitrate- or nitrite-forming compounds, e.g. nitrogen oxides and/or organic nitrogen compounds, are used together with an organic halide, in particular an organic chloride.

The reaction modifiers are generally effective when used in low concentration in the feed, for example up to 0.1 mole-%, relative to the total feed, for example from 0.01×10⁻⁴ to 0.01 mole-%. In particular when the olefin is ethylene, it is preferred that the reaction modifier is present in the feed at a concentration of at most 50×10⁻⁴ mole-%, in particular at most 20×10⁻⁴ mole-%, more in particular at most 15×10⁻⁴ mole-%, relative to the total feed, and preferably at least 0.2×10⁻⁴ mole-%, in particular at least 0.5×10⁻⁴ mole-%, more in particular at least 1×10⁻⁴ mole-%, relative to the total feed.

In addition to the olefin, oxygen, and the reaction modifier, the feed may contain one or more optional components, for example inert gases and saturated hydrocarbons. Inert gases, for example nitrogen or argon, may be present in the feed in a concentration of from 30 to 90 mole-%, typically from 40 to 80 mole-%, relative to the total feed. The feed may contain saturated hydrocarbons. Suitable saturated hydrocarbons are methane and ethane. If saturated hydrocarbons are present, they may be present in a quantity of up to 80 mole-%, relative to the total feed, in particular up to 75 mole-%. Frequently they may be present in a quantity of at least 30 mole-%, more frequently at least 40 mole-%. Saturated hydrocarbons may be added to the feed in order to increase the oxygen flammability limit.

The epoxidation process may be carried out using epoxidation conditions, including temperature and pressure, selected from a wide range. Frequently the reaction temperature is in the range of from 150 to 340° C., more frequently in the range of from 180 to 325° C. The reaction temperature may be increased gradually or in a plurality of steps, for example in steps of from 0.1 to 20° C., in particular 0.2 to 10° C., more in particular 0.5 to 5° C. The total increase in the reaction temperature may be in the range of from 10 to 140° C., more typically from 20 to 100° C. The reaction temperature may be increased typically from a level in the range of from 150 to 300° C., more typically from 200 to 280° C., when a fresh catalyst is used, to a level in the range of from 230 to 340° C., more typically from 240 to 325° C., when the catalyst has decreased in activity due to ageing.

The epoxidation process is typically carried out at a reactor inlet pressure in the range of from 1000 to 3500 kPa. “GHSV” or Gas Hourly Space Velocity is the unit volume of gas at normal temperature and pressure (0 C, 1 atm, i.e. 101.3 kPa) passing over one unit volume of packed catalyst per hour. Frequently, when the epoxidation process is a gas phase process involving a fixed catalyst bed, the GHSV is in the range of from 1500 to 10000 Nl/(l.h).

Carbon dioxide is a by-product in the epoxidation process, and thus may be present in the feed. The carbon dioxide may be present in the feed as a result of being recovered from the product mix together with unconverted olefin and/or oxygen and recycled. The term “product mix” as used herein is understood to refer to the product recovered from the outlet of the epoxidation reactor. Typically, a concentration of carbon dioxide in the feed in excess of 25 mole-%, preferably frequently in excess of 10 mole-%, relative to the total feed, is avoided. A preferred concentration of carbon dioxide in the feed is in the range of from 0.5 to 1 mole-% relative to the total feed. A process conducted in the absence of carbon dioxide in the feed, however, is within the scope of the present invention.

The olefin oxide produced may be recovered from the product mix by using methods known in the art, for example by absorbing the olefin oxide from a product mix in water and optionally recovering the olefin oxide from the aqueous solution by distillation. At least a portion of the aqueous solution containing the olefin oxide may be applied in a subsequent process for converting the olefin oxide into a 1,2-diol, a 1,2-diol ether, or an alkanolamine. The methods employed for such conversions are not limited, and those methods known in the art may be employed. The conversion into the 1,2-diol or the 1,2-diol ether may comprise, for example, reacting the olefin oxide with water, suitably using an acidic or a basic catalyst. For example, for making predominantly the 1,2-diol and less 1,2-diol ether, the olefin oxide may be reacted with a ten fold molar excess of water, in a liquid phase reaction in presence of an acid catalyst, e.g., 0.5-1.0% w sulfuric acid, based on the total reaction mixture, at 50-70° C. at 1 bar absolute, or in a gas phase reaction at 130-240° C. and 20-40 bar absolute, preferably in the absence of a catalyst. If the proportion of water is lowered, the proportion of 1,2-diol ethers is increased. The 1,2-diol ethers thus produced may be a di-ether, tri-ether, tetra-ether or a subsequent ether. Alternatively, 1,2-diol ethers may be prepared by converting the olefin oxide with an alcohol, in particular a primary alcohol, such as methanol or ethanol, by replacing at least a portion of the water by the alcohol.

The conversion into the alkanolamine may comprise reacting the olefin oxide with an amine, such as ammonia, an alkyl amine, or a dialkylamine. Anhydrous or aqueous ammonia may be used. Anhydrous ammonia is typically used to favor the production of monoalkanolamine. For methods applicable in the conversion of the olefin oxide into the alkanolamine, reference may be made to, for example U.S. Pat. No. 4,845,296, which is incorporated herein by reference.

The 1,2-diol and the 1,2-diol ether may be used in a large variety of industrial applications, for example in the fields of food, beverages, tobacco, cosmetics, thermoplastic polymers, curable resin systems, detergents, heat transfer systems, etc. The alkanolamine may be used, for example, in the treating (“sweetening”) of natural gas.

Unless specified otherwise, the organic compounds mentioned herein, for example the olefins, 1,2-diols, 1,2-diol ethers, alkanolamines, organic nitrogen compounds, and organic halides, have typically at most 40 carbon atoms, more typically at most 20 carbon atoms, in particular at most 10 carbon atoms, more in particular at most 6 carbon atoms. As defined herein, ranges for numbers of carbon atoms (i.e., carbon number) include the numbers specified for the limits of the ranges.

Having generally described the invention, a further understanding may be obtained by reference to the following examples, which are provided for purposes of illustration only and are not intended to be limiting unless otherwise specified.

### EXAMPLE 1

Formation of Carrier Particles

The transition alumina powder was obtained by digesting aluminum wire in a 3 wt. % acetic acid solution with stirring. During the digestion process, the temperature was maintained between 70° C. and 95° C. After about 30 hours, all the metal had been digested. The system was thereafter maintained at a temperature between 70° C. and 95° C. with stirring for an additional 3 days to increase the crystallinity. The alumina sol was then spray dried to obtain the transition alumina powder.

Transition alumina powder was combined with alumina sol, obtainable as described above, in a blender for 10 minutes to form an extrudable paste. The transition alumina powder and alumina sol (10% alumina by weight) were used in a weight ratio of 1000:730.

The paste was extruded into cylinders that were dried at 190° C. for 6 hours. The cylinders were then calcined at 600° C. for 60 minutes in a rotating calciner.

Fluoride Mineralization

An impregnation solution was made by dissolving 19.58 g of ammonium fluoride in 480 g of distilled water. The amount of ammonium fluoride was determined by:

$$F \times {m}_{alumina} [ \frac{wt \% {NH}_{4} F}{100 - wt \% {NH}_{4} F} ]$$

where F is a factor that is at least 1.5. The amount of water was determined by:
F×mₐₗᵤₘᵢₙₐ×WABS
where mₐₗᵤₘᵢₙₐ is the mass of the transition alumina starting material, wt % NH₄F is the weight percent of ammonium fluoride used, and WABS is the water absorption (g H₂O/g alumina) of the transition alumina. The factor “F” is large enough to provide an excess of impregnation solution that allows the alumina to be completely submerged.

320 grams of the transition alumina carrier cylinders obtained above were evacuated to 20 mm Hg for 3 minute and the final impregnating solution was added to the carrier cylinders while under vacuum. The vacuum was released and the carrier cylinders were allowed to contact the liquid for 5 minutes. The impregnated carrier cylinders were then centrifuged at 500 rpm for 2 minutes to remove excess liquid. Impregnated transition alumina cylinders were dried in flowing nitrogen at 120° C. for 10 hours.

The dried impregnated transition alumina carrier was then subjected to a calcination step. 25 grams of the dried impregnated transition alumina carrier cylinders were placed in a first high temperature alumina crucible. Approximately 50 g of calcium oxide was placed in a second high temperature alumina crucible that was of a greater diameter than the first crucible. The high temperature alumina crucible that contained the impregnated transition alumina carrier cylinders was placed into the second high temperature alumina crucible, which contained the calcium oxide, and was then covered with a third high temperature alumina crucible of smaller diameter than the second crucible and greater diameter than the first crucible, such that the impregnated transition alumina carrier cylinders alumina were locked in by the third crucible and the calcium oxide. This assembly was placed into a cool, room temperature furnace. The temperature of the furnace was increased from room temperature to 800° C. over a period of 30 minutes. The assembly was then held at 800° C. for 30 minutes and thereafter heated to 1200° C. over a period of 40 minutes. The assembly was then held at 1200° C. for 1 hour. The furnace was then allowed to cool and the alumina removed from the assembly.

The carrier thus obtained (Carrier A) had the properties described in Table 1. The carrier had a particulate matrix having a morphology characterizable as lamellar or platelet-type.

TABLE 1
Properties of Carrier Support

| | Carrier A |
|---|---|
| Properties | |
| Water Absorption (g/g) | 0.53 |
| Surface Area (m²/g) | 0.71 |

Catalyst Preparation

In a 5-liter stainless steel beaker, 415 grams of reagent grade sodium hydroxide was dissolved in 2340 mL of deionized water. The temperature of the solution was adjusted to about 50° C. In a 4-liter stainless steel beaker, 1699 grams of silver nitrate was dissolved in 2100 mL of deionized water. The temperature of the solution was adjusted to about 50° C. The sodium hydroxide solution was slowly added to the silver nitrate solution with stirring while the temperature was maintained at about 50° C. The resulting slurry was stirred for about 15 minutes. The pH of the solution was maintained at above 10 by the addition of NaOH solution as required. A washing procedure was used which included removing liquid by the use of a filter wand followed by the replacement of the removed liquid with an equivalent volume of deionized water. This washing procedure was repeated until the conductivity of the filtrate dropped below 90 micro-mho/cm. After the completion of the last wash cycle, 1500 mL of deionized water was added, followed by the addition of 630 grams of oxalic acid dihydrate (4.997 moles) in increments of 100 grams while stirring and maintaining the solution at about 40° C. (±5° C.). The pH of the solution was monitored during the addition of the last 130 grams of oxalic acid dihydrate to ensure that it did not drop below 7.8 for an extended period of time. Water was removed from the solution with a filter wand and the slurry was cooled to less than 30° C. Slowly added to the solution was 732 grams of 92% ethylenediamine. The temperature was maintained below 30° C. during this addition. A spatula was used to manually stir the mixture until enough liquid was present to mechanically stir. The final solution was used as a stock silver impregnation solution.

The impregnation solution for preparing Catalyst A was made by mixing 145.0 grams of stock silver solution of specific gravity 1.550 g/cc with a solution of 0.0944 g of NH₄ReO₄ (ammonium perrhenate) in ˜2 g of 1:1 EDA/H₂O (ethylenediamine/water), 0.0439 g of ammonium metatungstate dissolved in ˜2 g of 1:1 ammonia/water and 0.1940 g LiNO₃ (lithium nitrate) dissolved in water. Additional water was added to adjust the specific gravity of the solution to 1.507 g/cc. The doped solution was mixed with 0.0675 g of 44.62% CsOH (cesium hydroxide) solution. This final impregnating solution was used to prepare Catalyst A. 30 grams of Carrier A was evacuated to 20 mm Hg for 1 minute and the final impregnating solution was added to Carrier A while under vacuum, then the vacuum was released and the carrier allowed to contact the liquid for 3 minutes. The impregnated Carrier A was then centrifuged at 500 rpm for 2 minutes to remove excess liquid. Impregnated Carrier A pellets were placed in a vibrating shaker and dried in flowing air at 250° C. for 5.5 minutes. The final Catalyst A composition was 18.3% Ag, 400 ppm Cs/g catalyst, 1.5 μmole Re/g catalyst, 0.75 μmole W/g catalyst, and 12 μmole Li/g catalyst.

Catalyst Testing

Catalyst A was used to produce ethylene oxide from ethylene and oxygen. To do this, 3.829 g of crushed Catalyst A was loaded into a stainless steel U-shaped tube. The tube was then immersed in a molten metal bath (heat medium) and the ends were connected to a gas flow system. The weight of catalyst used and the inlet gas flow rate were adjusted to give a gas hourly space velocity of 3300 Nl/(l.h), as calculated for uncrushed catalyst. The gas flow was adjusted to 16.9 Nl/h. The inlet gas pressure was 1370 kPa.

The gas mixture passed through the catalyst bed, in a “once-through” operation, during the entire test run including the start-up, was 30% v ethylene, 8% v oxygen, 2.0% v carbon dioxide, 61.5% v nitrogen and 2.0 to 6.0 parts by million by volume (ppmv) ethyl chloride.

For Catalyst A, the initial reactor temperature was 190° C., which was ramped up at a rate of 10° C. per hour to 220° C. and then adjusted so as to achieve a desired constant level of ethylene oxide production, conveniently measured as partial pressure of ethylene oxide at the reactor outlet or molar percent ethylene oxide in the product mix.

At an ethylene oxide production level of 41 kPa for ethylene oxide partial pressure, Catalyst A provided an initial selectivity of as much as about 90.4% at a temperature of 250° C. The catalyst selectivity remained above 87% until a cumulative ethylene oxide production of 0.62 kT/m³ had been achieved.

### COMPARATIVE EXAMPLE

Carrier

AX300, a commercial gamma alumina extrudate available from Criterion and not prepared in accordance with the present invention, was used.

Fluoride Mineralization

An impregnation solution was made by dissolving 14.14 g of ammonium fluoride in 485.1 g of distilled water, with the amount of ammonium fluoride and the amount of distilled water being determined as described in Example 1.

231 grams of AX300 gamma alumina extrudate were evacuated to 20 mm Hg for 3 minutes and the final impregnating solution was added to the carrier cylinders while under vacuum. The vacuum was released and the carrier cylinders were allowed to contact the liquid for 5 minutes. The impregnated carrier cylinders were then centrifuged at 500 rpm for 2 minutes to remove excess liquid. Impregnated transition alumina cylinders were dried in flowing nitrogen at 120° C. for 10 hours.

25 grams of the dried impregnated transition alumina carrier cylinders thus obtained were subjected to the calcinations procedure described in Example 1.

The carrier thus obtained (Carrier B) had the properties described in Table 2. The carrier had a particulate matrix having a morphology characterizable as lamellar or platelet-type.

TABLE 2
Properties of Carrier Support

| | Carrier B |
|---|---|
| Properties | |
| Water Absorption (g/g) | 0.70 |
| Surface Area (m²/g) | 0.75 |

Catalyst Preparation

The stock silver impregnation solution described in Example 1 was used to prepare Catalyst B. The impregnation solution for preparing Catalyst B was made by mixing 145.0 grams of the stock silver solution with a solution of 0.0756 g of NH₄ReO₄ (ammonium perrhenate) in ˜2 g of 1:1 EDA/H₂O (ethylenediamine/water), 0.0352 g of ammonium metatungstate dissolved in ˜2 g of 1:1 ammonia/water and 0.1555 g LiNO₃ (lithium nitrate) dissolved in water. Additional water was added to adjust the specific gravity of the solution to 1.507 g/cc. The doped solution was mixed with 0.0406 g of 45.4% CsOH (cesium hydroxide) solution. This final impregnating solution was used to prepare Catalyst B. 30 grams of Carrier B was evacuated to 20 mm Hg for 1 minute and the final impregnating solution was added to Carrier B while under vacuum, then the vacuum was released and the carrier allowed to contact the liquid for 3 minutes. The impregnated Carrier B was then centrifuged at 500 rpm for 2 minutes to remove excess liquid. Impregnated Carrier B pellets were placed in a vibrating shaker and dried in flowing air at 250° C. for 5.5 minutes. The final Catalyst B composition was 22.83% Ag, 300 ppm Cs/g catalyst, 1.5 μmole Re/g catalyst, 0.75 μmole W/g catalyst, and 12 μmole Li/g catalyst.

Catalyst Testing

Catalyst B was used to produce ethylene oxide from ethylene and oxygen. To do this, 2.58 g of crushed Catalyst B was loaded into a stainless steel U-shaped tube. The tube was then immersed in a molten metal bath (heat medium) and the ends were connected to a gas flow system. The weight of catalyst used and the inlet gas flow rate were adjusted to give a gas hourly space velocity of 3300 Nl/(l.h), as calculated for uncrushed catalyst. The gas flow was adjusted to 16.9 Nl/h. The inlet gas pressure was 1370 kPa.

The gas mixture passed through the catalyst bed, in a “once-through” operation, during the entire test run including the start-up, was 30% v ethylene, 8% v oxygen, 2.0% v carbon dioxide, 61.5% v nitrogen and 2.0 to 6.0 parts by million by volume (ppmv) ethyl chloride.

For Catalyst B, the initial reactor temperature was 190° C., which was ramped up at a rate of 10° C. per hour to 220° C. and then adjusted so as to achieve a desired constant level of ethylene oxide production. At an ethylene oxide production level of 41 kPa for ethylene oxide partial pressure, Catalyst B provided an initial selectivity of as much as about 88.4% at a temperature of 268° C. The catalyst selectivity remained above 87% until a cumulative ethylene oxide production of 0.16 kT/m³ had been achieved.

We claim:

1. A carrier comprising alpha-alumina obtainable from a process comprising acid digestion of aluminum.

2. A method of preparing a carrier comprising:
a. acid digesting aluminum to obtain transition alumina;
b. forming a paste comprising the transition alumina; and
c. forming carrier particles comprising transition alumina from the paste.

3. The method as claimed in claim 2 wherein step (a) comprises the steps of acid digesting aluminum to obtain an alumina sol, and converting alumina sol to transition alumina powder.

4. The method as claimed in claim 3 wherein the paste is formed from a mixture comprising alumina sol and the transition alumina powder.

5. The method as claimed in claim 2 further comprising:
d. calcining the carrier particles at a temperature between 900° C. and 1400° C.

6. The method as claimed in claim 2 wherein the aluminum comprises aluminum wire.

7. The method as claimed in claim 2 wherein the acid comprises acetic acid.

8. The method as claimed in claim 2 wherein the method additionally comprises incorporating a fluorine-containing species in the carrier.

9. A carrier comprising alpha-alumina, which carrier is obtainable from the method of claim 2.

10. A carrier comprising alpha-alumina obtainable from a process comprising:
a. acid digesting aluminum to obtain an alumina sol;
b. forming transition alumina powder from the alumina sol;
c. forming a paste with the transition alumina powder;
d. forming carrier particles comprising transition alumina from the paste; and
e. calcining the carrier particles at a temperature between 900° C. and 1400° C.

11. The carrier as claimed in claim 10 wherein the paste is formed from a mixture comprising alumina sol and the transition alumina powder.

12. The carrier as claimed in claim 10 wherein the paste is formed from a mixture comprising alumina sol formed in step (a) and the transition alumina powder.

13. The carrier as claimed in claim 11 wherein the weight ratio of transition alumina powder to alumina sol is from 1000:500 to 1000:850.

14. The carrier as claimed in claim 10 wherein the carrier is a fluoride mineralized carrier and wherein the carrier particles are calcined at a temperature between 900° C. and 1200° C.

15. The carrier as claimed in claim 10 wherein the carrier comprises a particulate matrix having a morphology characterizable as lamellar.

16. A catalyst for the epoxidation of an olefin comprising a silver component deposited on a carrier comprising alpha-alumina, wherein the carrier is obtainable from a process comprising acid digestion of aluminum.

17. A catalyst for the epoxidation of an olefin comprising a silver component deposited on a carrier according to claim 10.

18. The catalyst as claimed in claim 16, wherein the catalyst additionally comprises a high selectivity dopant.

19. A catalyst as claimed in claim 16, wherein the catalyst additionally comprises a Group IA metal component.

20. A catalyst as claimed in claim 16, wherein the catalyst additionally comprises a rhenium component, or a rhenium component and a rhenium co-promoter.

21. A process for the epoxidation of an olefin comprising the steps of:
contacting a feed comprising an olefin and oxygen with a catalyst comprising a silver component deposited on a carrier comprising alpha-alumina; and
producing a product mix comprising an olefin oxide, wherein the carrier is obtained from a process comprising acid digestion of aluminum.

22. A process for the epoxidation of an olefin comprising the steps of:
contacting a feed comprising an olefin and oxygen with a catalyst comprising a silver component deposited on a carrier in accordance with claim 10;
and producing a product mix comprising an olefin oxide.

23. The process as claimed in claim 21, wherein the catalyst additionally comprises a high selectivity dopant.

24. The process as claimed in claim 21, wherein the catalyst additionally comprises a Group IA metal component.

25. The process as claimed in claim 21, wherein the catalyst additionally comprises a rhenium component, or a rhenium component and a rhenium co-promoter.

26. The process as claimed in claim 21, wherein the olefin comprises ethylene.

27. A process for the production of a 1,2-diol, a 1,2-diol ether or an alkanolamine comprising converting an olefin oxide into the 1,2-diol, the 1,2-diol ether or the alkanolamine wherein the olefin oxide has been obtained by a process for the epoxidation of an olefin as claimed in claim 21.

28. A carrier as claimed in claim 10, which carrier is suitable for use as a carrier of a catalyst for use in a process for the epoxidation of an olefin.
